(19) United States
(12) Patent Application Publication
(10) Pub. No.: US 2007/0086969 A1
(43) Pub. Date: Apr. 19, 2007

(54) Miracle hair treatment

(76) Inventor: Eula Palmer, Brooklyn, NY (US)

Correspondence Address: JAMES RAY & ASSOCIATES, 2640 PITCAIRN ROAD, MONROEVILLE, PA, 15146, US

(21) Appl. No.: 11/251,194

(22) Filed: Oct. 14, 2005

(51) Int. Cl. A61K 8/97; A61K 8/64; A61K 36/23; A61K 36/63

(52) U.S. Cl. 424/70.14; 424/74; 424/769; 424/773

(57) ABSTRACT

A composition for the treatment of hair. The composition consists essentially of mayonnaise being present in the composition in a concentration of between about 55 and 64 percent by weight. Olive oil is present in the composition in a concentration of between about 12 and 15 percent by weight, carrot oil is present in the composition in a concentration of between about 12 and 15 percent by weight, and collagen is present in the composition in a concentration of between about 12 and 15 percent by weight.

### FIELD OF THE INVENTION

The present invention relates, in general, to a composition for hair treatment, and more specifically, the present invention relates to a new formulation for treatment of brittle and thinning hair and a process for such treatment of problem hair.

### BACKGROUND OF THE INVENTION

There are many products on the market for hair treatment. From tonics and gels as an aid in combing hair and conditioners for softening hair or making the hair more manageable to such products that claim hair restoration for bald spots the number of products are endless.

Many of these products deal with one specific type of hair or scalp disorder be it itching scalp, thinning hair, brittle hair, dry hair, oily hair or whatever. So a person may end up with a variety of products that accomplish what the consumer desires. Thus, it would be desirable if one product could solve many of the disorders rather than requiring a product for each disorder.

### SUMMARY OF THE INVENTION

The present invention provides a composition for the treatment of hair. The composition consists essentially of mayonnaise being present in the composition in a concentration of between about 55 and 64 percent by weight. Olive oil is present in the composition in a concentration of between about 12 and 15 percent by weight, carrot oil is present in the composition in a concentration of between about 12 and 15 percent by weight, and collagen is present in the composition in a concentration of between about 12 and 15 percent by weight.

An alternate embodiment of the invention provides a method for the treatment of hair disorders. The method comprises the steps of washing the hair to be treated with a predetermined shampoo and then the step of rinsing the hair to remove traces of the shampoo. There is a step of drying the hair and a step of depositing a predetermined amount of a predetermined hair formulation onto the hair and distributing such formulation throughout the hair. Further, there is a step of covering the hair with a cap for at least 30 minutes and drying the hair with a hair dryer for a predetermined time. Another step involves rinsing the hair with lukewarm water and a step of adding a desired conditioning rinse to the hair.

### OBJECTS OF THE INVENTION

It is, therefore, one of the primary objects of the present invention to provide a hair treatment that can strengthen hair roots thus making the hair healthier.

Another object of the present invention is to provide a hair treatment that can prevent dry scalp.

Yet another object of the present invention is to provide a hair treatment that can promote new growth in bald spots. Still another object of the present invention is to provide a process for the use of such hair treatment.

These and various other objects and advantages of this invention will become apparent after a full reading of the following detailed description, particularly, when read in conjunction with the appended claims.

### BRIEF DESCRIPTION OF THE PRESENTLY PREFERRED AND ALTERNATE EMBODIMENTS OF THE INVENTION

The present invention eliminates many of the problems encountered in hair treatments. Thus, the present invention provides a hair treatment that will encourage hair growth, strengthen hair roots, soften dry, brittle hair and help prevent dry scalp.

The hair treatment comprises a blend of oil, mayonnaise, carrot oil and collagen. In a presently preferred embodiment of the invention it preferred that such oil is olive oil.

Such formulation for such hair treatment includes mayonnaise being present in such formulation in a concentration of between about 55 to about 65 percent by weight. It is presently preferred that such mayonnaise is present in a concentration of between about 58 to about 62 percent by weight.

Such hair formulation includes olive oil. Such olive oil is present in a concentration of between about 12 and about 15 percent by weight. In a preferred embodiment of the invention such olive oil is present in a concentration of between about 13 and 14 percent by weight.

Such hair formulation includes carrot oil. Such carrot oil is present in a concentration of between about 12 and about 15 percent by weight. In a preferred embodiment of the invention such carrot oil is present in a concentration of between about 13 and 14 percent by weight.

The hair formulation further includes collagen. Such collagen is present in a concentration of between about 12 and about 15 percent by weight. In a preferred embodiment of the invention such collagen is present in a concentration of between about 13 and 14 percent by weight. Collagen is an insoluble fibrous protein that occurs as a chief constituent of connective tissue fibrils and in bones and forms a gelatinous or glue like material on prolonged heating with water.

An alternate embodiment of the invention provides a method for the treatment of hair disorders. The method comprises the step of washing the hair to be treated with a predetermined shampoo and then the step of rinsing the hair to remove traces of the shampoo. There is a step of drying the hair, drying such hair can be just towel drying, and a step of depositing a predetermined amount of a predetermined hair formulation onto the hair and distributing such formulation throughout the hair. Further, there is a step of covering the hair with a cap for at least 30 minutes and drying the hair with a hair dryer for a predetermined time. Another step involves rinsing the hair with lukewarm water and a step of adding a desired conditioning rinse to the hair.

The method further includes the step of styling said hair after the hair has been treated with a desired conditioning rinse. It is also presently preferred that distributing such predetermined formulation throughout such hair is done by combing the hair.

It is presently preferred that the drying of the hair after the hair has been covered with a cap is performed in a hood type hair dryer such as is normally found in a salon. It is preferred that such drying be for twenty minutes for other than a first time user. It is also presently preferred that such drying be extended to thirty minutes for a first time user of the method. It is also presently preferred that such cap used to cover the hair is a plastic cap.

It is preferred that such predetermined formulation distributed through such hair includes a blend of mayonnaise, olive oil, carrot oil and collagen. Such blend has been described previously.

While both the presently preferred and a number of alternative embodiments of the present invention have been described in detail above it should be understood that various other adaptations and modifications of the present invention can be envisioned by those persons who are skilled in the relevant art without departing from either the spirit of the invention or the scope of the appended claims.

I claim:

1. A composition for treatment of hair, said composition consisting essentially of:
(a) mayonnaise being present in said composition in a concentration of between about 55 and 64 percent by weight;
(b) olive oil being present in said composition in a concentration of between about 12 and 15 percent by weight;
(c) carrot oil being present in said composition in a concentration of between about 12 and 15 percent by weight; and
(d) collagen being present in said composition in a concentration of between about 12 and 15 percent by weight.

2. The composition, according to claim 1, wherein said mayonnaise is present in a concentration of between about 58 and about 62 percent.

3. The composition, according to claim 1, wherein said olive oil is present in a concentration of between about 13 and about 14 percent.

4. The composition, according to claim 1, wherein said carrot oil is present in a concentration of between about 13 and about 14 percent.

5. The composition, according to claim 1, wherein said collagen is present in a concentration of between about 13 and about 14 percent.

6. A method for treatment of hair disorders; said method comprising the step of:
(a) washing said hair to be treated with a preselected shampoo;
(b) rinsing said hair to remove traces of said shampoo;
(c) drying said hair;
(d) depositing a predetermined amount of a predetermined hair formulation onto said hair;
(e) distributing said formulation throughout said hair;
(f) covering said hair with a cap for at least 30 minutes;
(g) drying said hair with a hair dryer for a predetermined time;
(h) rinsing said hair with lukewarm water; and
(i) adding a desired conditioning rinse to said hair.

7. The method according to claim 6, wherein said method further includes styling said hair after step (i).

8. The method according to claim 6, wherein said method includes use of a hood type hair dryer in step (g).

9. The method according to claim 6, wherein said method includes towel drying in step (c).

10. The method according to claim 6, wherein said predetermined time for drying said hair in step (g) is for at least thirty minutes for a first time user of said method.

11. The method according to claim 6, wherein said predetermined time for drying said hair in step (g) is for twenty minutes for other than first time users of said method.

12. The method according to claim 6, wherein said predetermined formulation distributed through said hair in step (e) includes a blend of mayonnaise, olive oil, carrot oil and collagen.

13. The method, according to claim 12, wherein said mayonnaise is present in a concentration of between about 55 and about 64 percent by weight, said olive oil is present in a concentration of between about 12 and about 15 percent, said carrot oil is present in a concentration of between about 12 and about 15 percent and said collagen is present in a concentration of between about 12 and about 15 percent in said predetermined formulation.

14. The method, according to claim 13, wherein said mayonnaise is present in a concentration of between about 58 and about 62 percent by weight, said olive oil is present in a concentration of between about 13 and about 14 percent, said carrot oil is present in a concentration of between about 13 and about 14 percent and said collagen is present in a concentration of between about 13 and about 14 percent in said predetermined formulation.

15. The method according to claim 6, wherein said cap for covering said hair in step (f) is plastic.

16. The method according to claim 6, wherein said method includes combing said hair in step (e) to distribute said formulation throughout said hair.
